(19) United States
(12) Patent Application Publication
(10) Pub. No.: US 2018/0098703 A1
(43) Pub. Date: Apr. 12, 2018

(54) DISPOSABLE EAR PROBE COVER FOR THE SAME, AND EAR PROBE COVER SET

(71) Applicant: Quanta Computer Inc., Taoyuan City (TW)

(72) Inventors: Wan-Chi LIN, Taoyuan City (TW); Yun-Cheng LIU, Taoyuan City (TW)

(73) Assignee: Quanta Computer Inc., Taoyuan City (TW)

(21) Appl. No.: 15/373,592

(22) Filed: Dec. 9, 2016

(30) Foreign Application Priority Data
Oct. 6, 2016 (TW) 105215208

(51) Int. Cl. A61B 5/01; A61B 5/00; A61B 50/30; B65D 85/62

(52) U.S. Cl. CPC A61B 5/01; A61B 5/0086; A61B 5/6817; A61B 2562/242; B65D 85/62; A61B 2562/247; A61B 50/30

(57) ABSTRACT

A disposable ear probe cover includes a light-transmissive cover body, a flange and a spacer member. The flange is formed on the light-transmissive cover body to surround an opening end of the light-transmissive cover. The spacer member is formed on the light-transmissive cover body. Therefore, when the disposable ear probe cover is sleeved into another disposable ear probe cover which is identical to the disposable ear probe cover, the spacer member of the disposable ear probe cover supports the other disposable ear probe cover so that a void gap is defined between the flange of the disposable ear probe cover and another flange of the another disposable ear probe cover.

### RELATED APPLICATIONS

This application claims priority to Taiwan Application Serial Number 105215208, filed Oct. 6, 2016, which is herein incorporated by reference.

### BACKGROUND

### Field of Disclosure

The present disclosure relates to an ear probe cover. More particularly, the present disclosure relates to a disposable ear probe cover for an infrared ear thermometer.

### Description of Related Art

In general, since an infrared ear thermometer can be used to respectively measure body temperatures within ears of different measured persons, thus, before measuring the body temperature of one of the measured persons, a probe head of the infrared ear thermometer must be covered by a disposable probe cover in order to keep the probe head clean and reducing cross-infection between the measured persons.

However, when the conventional disposable probe covers are directly stacked with each other, there is no gap existing between any two of the conventional disposable probe covers. Thus, when a user desires to put a clean disposable probe cover from the stacked disposable probe covers on the probe head of the infrared ear thermometer, the user has to directly touch the disposable probe cover with the hands (e.g., nails) of the user for removing the clean disposable probe cover away from the remaining disposable probe covers. Thereby, increasing the possibility of the clean disposable probe cover becoming contaminated or prolonging the replacement time of the disposable probe covers from the infrared ear thermometer.

### SUMMARY

An aspect of the disclosure is to provide a disposable ear probe cover for the same, and an ear probe cover set formed by a plurality of the disposable ear probe covers which are sleeved to one another. Each of the disposable ear probe covers can be removed without directly touching the disposable ear probe cover with the hands of the user. Thereby, not only the possibilities of the disposable ear probe covers getting contaminated can be reduced, but also the replacement procedure for an infrared ear thermometer to change the disposable ear probe cover can be simplified, and the replacement time of the disposable ear probe cover can be decreased so as to overcome the defects and inconveniences of the prior art.

According to one embodiment, an ear probe cover set includes a container and a superimposed-cover structure received in the container, shaped as a tubular body formed by a plurality of disposable ear probe covers which are sleeved one another. Each of the disposable ear probe covers includes a light-transmissive cover body, a flange and a spacer member. The light-transmissive cover body is provided with an ear-entering end and an opening end which are opposite to each other. The flange is formed on the light-transmissive cover body and surrounds the opening end of the light-transmissive cover. The spacer member is formed on the light-transmissive cover body. Therefore, in any two neighboring ones of the disposable ear probe covers of the superimposed-cover structure, the spacer member of one of the two neighboring disposable ear probe covers supports the other of the two neighboring disposable ear probe covers so that a void gap can be defined between the flanges of the two neighboring disposable ear probe covers.

According to one embodiment, the disposable ear probe cover includes a light-transmissive cover body, a flange and a spacer member. The light-transmissive cover body is provided with an ear-entering end and an opening end which are opposite to each other. The flange is formed on the light-transmissive cover body and surrounds the opening end of the light-transmissive cover. The spacer member is formed on the light-transmissive cover body. Therefore, when the disposable ear probe cover is sleeved into another disposable ear probe cover which is identical to the disposable ear probe cover, the spacer member of the disposable ear probe cover supports the other disposable ear probe cover so that a void gap is defined between the flange of the disposable ear probe cover and another flange of the other disposable ear probe cover.

According to one embodiment, the disposable ear probe cover includes a light-transmissive cover body, a first surrounding portion and a second surrounding portion. The first surrounding portion is protrusively formed on one end of the light-transmissive cover body, and surrounds the light-transmissive cover body. The second surrounding portion is protrusively formed the light-transmissive cover body, located between the first surrounding portion and the other end of the light-transmissive cover body, and surrounds the light-transmissive cover body. Therefore, when the disposable ear probe cover is sleeved into another disposable ear probe cover which is identical to the disposable ear probe cover, the second surrounding portion of the disposable ear probe cover separates the first surrounding portion of the disposable ear probe cover and another first surrounding portion of the other disposable ear probe cover.

It is to be understood that both the foregoing general description and the following detailed description are by examples, and are intended to provide further explanation of the disclosure as claimed.

### BRIEF DESCRIPTION OF THE DRAWINGS

The accompanying drawings are included to provide a further understanding of the disclosure, and are incorporated in and constitute a part of this specification. The drawings illustrate embodiments of the disclosure and, together with the description, serve to explain the principles of the disclosure. In the drawings,

FIG. 1 is a schematic view showing an ear probe cover set according to one embodiment of the disclosure;

FIG. 2 is a partial cross-sectional view of two-neighboring disposable ear probe covers as taken along line A-A of FIG. 1;

FIG. 3A is a longitudinal sectional view of two sleeved disposable ear probe covers according to one embodiment of the disclosure;

FIG. 3B is a longitudinal sectional view of two sleeved disposable ear probe covers according to one embodiment of the disclosure;

FIG. 4 is a perspective view showing an infrared ear thermometer according to one embodiment of the disclosure;

FIG. 5 is a partial cross-sectional view of the infrared ear thermometer as taken along line B-B of FIG. 4;

FIG. 6A is an operational schematic view of the infrared ear thermometer of FIG. 4 pushing the clasping parts away; and

FIG. 6B is an operational schematic view of the infrared ear thermometer of FIG. 4 holding one of the disposable ear probe covers.

### DESCRIPTION OF THE EMBODIMENTS

Reference will now be made in detail to the present embodiments of the disclosure, examples of which are illustrated in the accompanying drawings. Wherever possible, the same reference numbers are used in the drawings and the description to refer to the same or like parts. According to the embodiments, it will be apparent to those skilled in the art that various modifications and variations can be made to the structure of the present disclosure without departing from the scope or spirit of the disclosure.

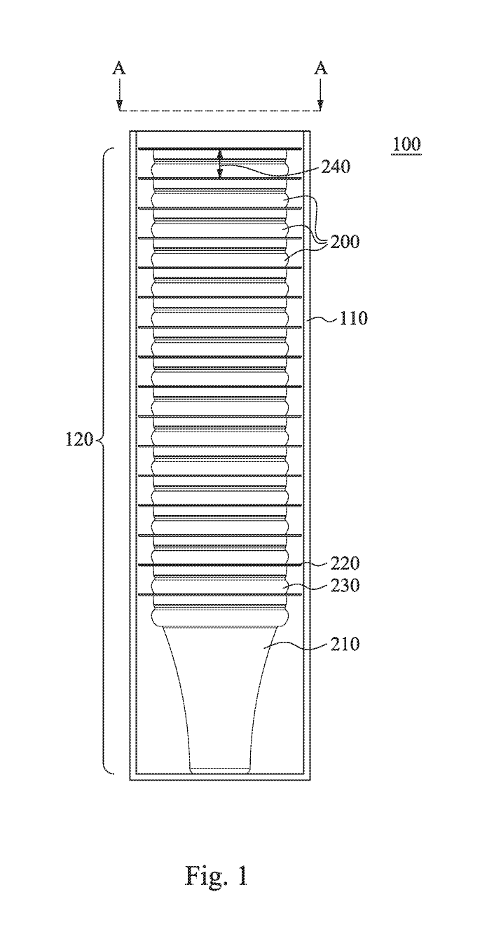

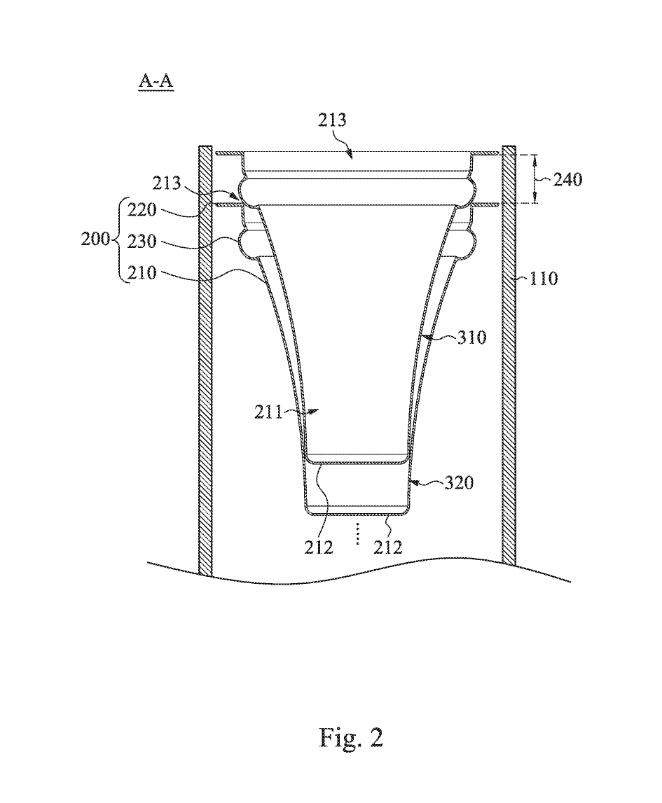

Reference is now made to FIG. 1 and FIG. 2, in which FIG. 1 is a schematic view showing an ear probe cover set 100 according to one embodiment of the disclosure, and FIG. 2 is a partial cross-sectional view of two-neighboring disposable ear probe covers as taken along line A-A of FIG. 1. As shown in FIG. 1 and FIG. 2, the ear probe cover set 100 includes a container 110 and a superimposed-cover structure 120 received in the container 110. The superimposed-cover structure 120 is shaped as a tubular body, and the tubular body is formed by a plurality of disposable ear probe covers 200 which are sleeved one another. Each of the disposable ear probe covers 200 can individually sleeve on an infrared ear thermometer, and each of the disposable ear probe covers 200 includes a light-transmissive cover body 210, a flange 220 and a spacer member 230. The light-transmissive cover body 210 is provided with an internal compartment 211, an ear-entering end 212 and an opening end 213 which is opposite to the ear-entering end 212. The ear-entering end 212 and the opening end 213 are respectively disposed on two opposite ends of the light-transmissive cover body 210, and the area of the ear-entering end 212 is smaller than the area of the opening end 213. The internal compartment 211 is defined in the light-transmissive cover body 210, and is connected to the ear-entering end 212 and the opening end 213. A probe head of the infrared ear thermometer inserts into the internal compartment 211 to reach the ear-entering end 212 inside the light-transmissive cover body 210 from the opening end 213 thereof. The ear-entering end 212 of the light-transmissive cover body 210 is closed for entering into an ear canal with the probe head of the infrared ear thermometer. The flange 220 is disposed on the opening end 213 of the light-transmissive cover body 210, and is extended outwards from the opening end 213, and the flange 220 surrounds the opening end 213 of the light-transmissive cover body 210. In this embodiment, since the flange 220 has a shape similar to a flake, and the flange 220 is continuous to surround the opening end 213, thus, the flange 220 can be told as one surrounding portion of the disposable ear probe cover 200.

The spacer member 230 is protrusively formed on the light-transmissive cover body 210. In this embodiment, the spacer member 230 is protrusively formed on the outer surface of the light-transmissive cover body 210, and is arranged between the ear-entering end 212 and the opening end 213. More particularly, the spacer member 230 is located on a position of the outer surface of the light-transmissive cover body 210 which is approximate to the flange 220.

In this embodiment, the spacer member 230 of the disposable ear probe cover 200 is circular ring-shaped, and continuously surrounds the light-transmissive cover body 210 so that the spacer member 230 can be told as another surrounding portion arranged on the outer surface of the light-transmissive cover body 210. Furthermore, a cross-section of the spacer member 230 also can be provided with an arc-profile; however, the disclosure is not limited to this shape.

In this embodiment, the light-transmissive cover body 210, the flange 220 and the spacer member 230 of each of the disposable ear probe cover 200 are integrally formed together; however, the disclosure is not limited thereto.

As shown in FIG. 2, in two neighboring ones of the disposable ear probe covers 200 of the superimposed-cover structure 120, since the spacer member 230 of the one disposable ear probe cover 200 directly abuts and supports the other disposable ear probe cover 200 so that a void gap 240 can be defined between the flanges 220 of the two neighboring disposable ear probe covers 200.

Specifically, in order to clearly comprehend the disposable ear probe covers 200 of FIG. 2, refer to FIG. 2, the two neighboring disposable ear probe covers 200 of FIG. 2 are tentatively named as a first ear probe cover 310 and a second ear probe cover 320. The ear-entering end 212 of the first ear probe cover 310 enters into the internal compartment 211 of the second ear probe cover 320 from the opening end 213 of the second ear probe cover 320, that is, the first ear probe cover 310 is partially received inside the second ear probe cover 320. Also, the spacer member 230 of the first ear probe cover 310 abuts to support the flange 220 of the second ear probe cover 320, thus, the void gap 240 can be formed between the flange 220 of the first ear probe cover 310 and the flange 220 of the second ear probe cover 320.

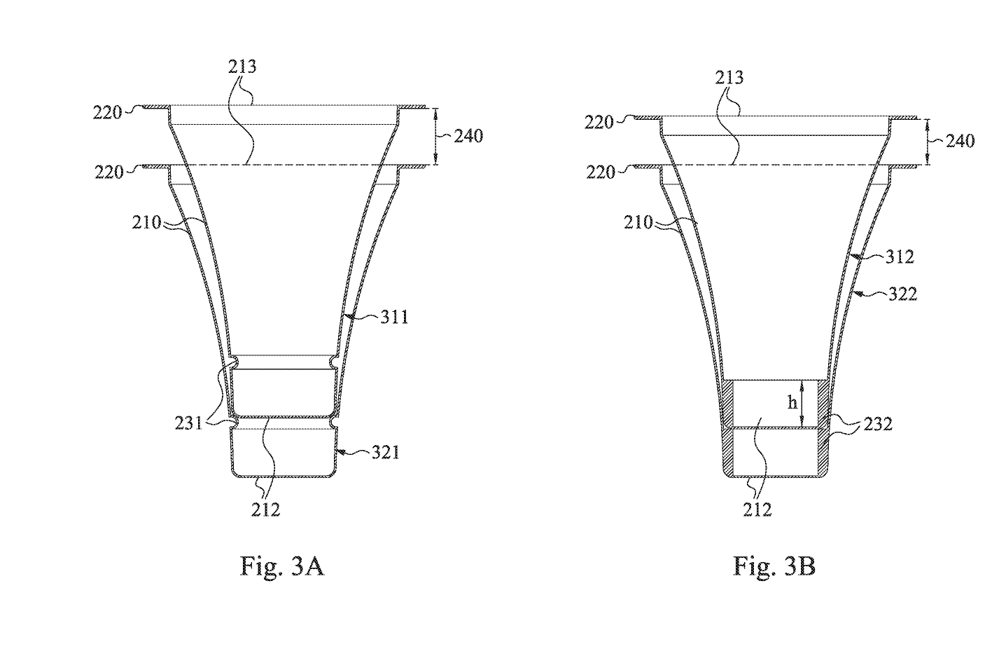

FIG. 3A is a longitudinal sectional view of two sleeved disposable ear probe covers according to one embodiment of the disclosure. As shown in FIG. 3A, each of the disposable ear probe covers of FIG. 3A is substantially the same as each of the disposable ear probe covers of FIG. 1, except that each of the spacer members 231 in this embodiment is concavely formed on the light-transmissive cover body 210, and is arranged between the ear-entering end 212 and the opening end 213 of the light-transmissive cover body 210. More particularly, each of the spacer members 231 of the ear probe covers 311 and 321 is protrusively formed on an inner surface of the light-transmissive cover body 210 being proximate to the ear-entering end 212.

In greater details, the two neighboring disposable ear probe covers of FIG. 3A are tentatively named as a first ear probe cover 311 and a second ear probe cover 321. The ear-entering end 212 of the first ear probe cover 311 enters into the internal compartment 211 of the second ear probe cover 321 from the opening end 213 of the second ear probe cover 321, that is, the first ear probe cover 311 is partially received inside the second ear probe cover 321, and the spacer member 231 of the second ear probe cover 321 abuts and supports the ear-entering end 212 of the first ear probe cover 311. Therefore, the void gap 240 can be formed between the flange 220 of the first ear probe cover 311 and the flange 220 of the second ear probe cover 321.

FIG. 3B is a longitudinal sectional view of two sleeved disposable ear probe covers according to one embodiment of the disclosure. As shown in FIG. 3B, as shown in FIG. 3B, each of the disposable ear probe covers of FIG. 3B is substantially the same as each of the disposable ear probe covers of FIG. 1, except that each of the spacer members 232 in this embodiment is protrusively formed on the inner surface of light-transmissive cover body 210 thereof, and is directly connected to the ear-entering end 212.

In greater details, the two neighboring disposable ear probe covers of FIG. 3B are tentatively named as a first ear probe cover 312 and a second ear probe cover 322. The ear-entering end 212 of the first ear probe cover 312 enters into the internal compartment 211 of the second ear probe cover 322 from the opening end 213 of the second ear probe cover 322, that is, the first ear probe cover 312 is partially received inside the second ear probe cover 322. Also, the spacer member 232 of the second ear probe cover 322 abuts and supports the ear-entering end 212 of the first ear probe cover 312, thus, the void gap 240 can be formed between the flange 220 of the first ear probe cover 312 and the flange 220 of the second ear probe cover 322, and the height h of the spacer member 232 is same as the height of the void gap 240.

It is noted, although the spacer member in all of the embodiments above continuously surrounds the light-transmissive cover body with a single-body structure, however, the spacer member in another embodiment in the disclosure also might include a plurality of bumps (not shown in figures) which are separately arranged on the outer surfaces light-transmissive cover body for surrounding the light-transmissive cover body.

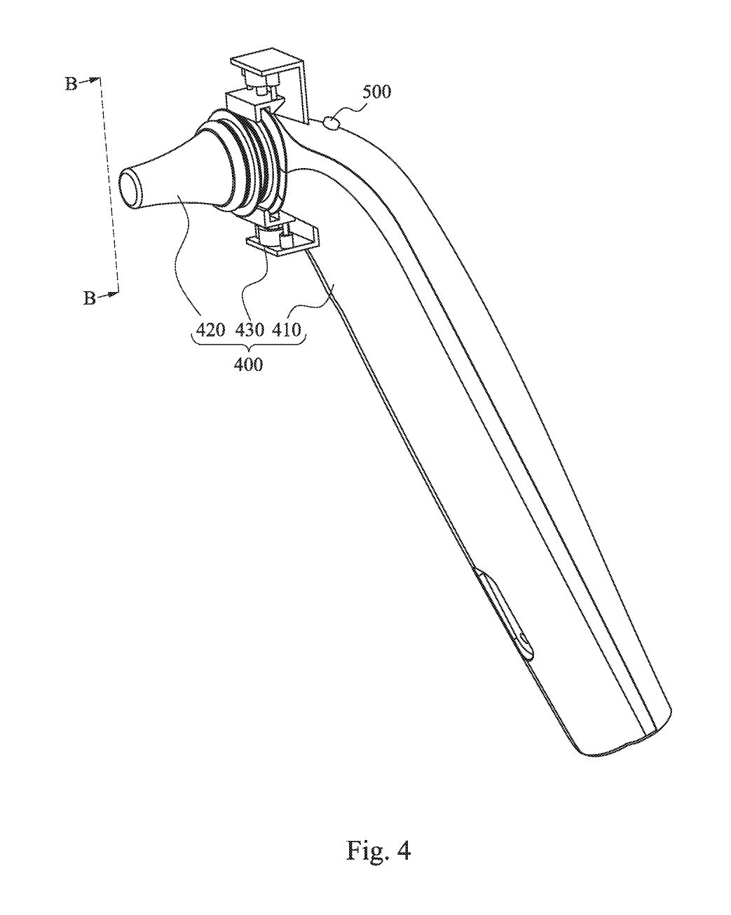

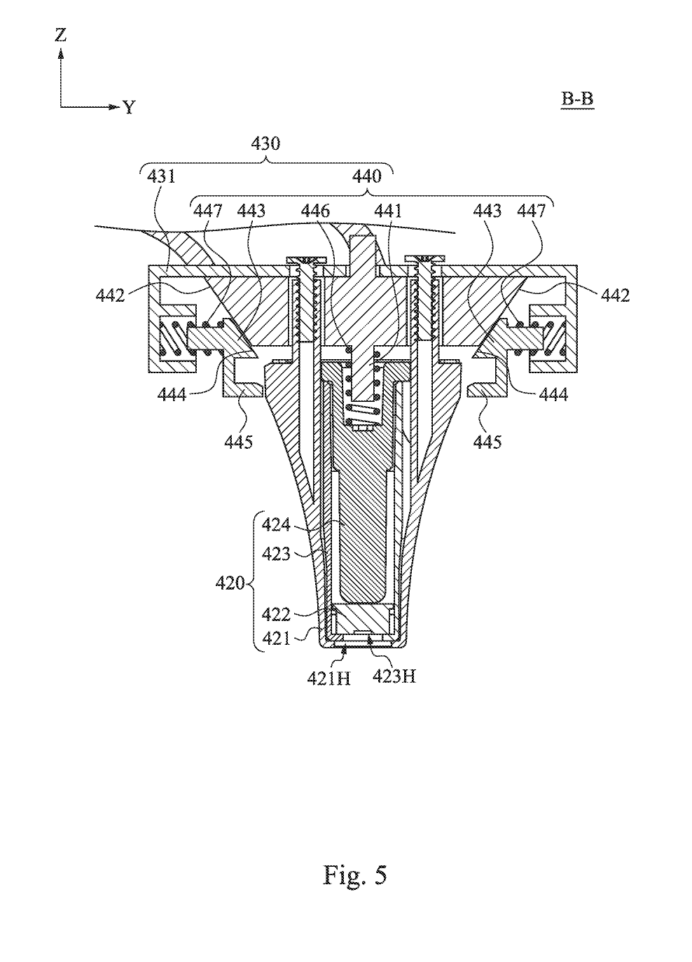

Reference is now made to FIG. 4 and FIG. 5, in which FIG. 4 is a perspective view showing an infrared ear thermometer 400 according to one embodiment of the disclosure, and FIG. 5 is a partial cross-sectional view of the infrared ear thermometer as taken along line B-B of FIG. 4. As shown in FIG. 4 and FIG. 5, the infrared ear thermometer 400 includes a main body 410, a probe head 420 and a detaching module 430. The main body 410 includes other known structure of a conventional infrared ear thermometer such as a handle part. The probe head 420 is connected to one end of the main body 410. A cross-sectional area of the probe head 420 has a shape of a truncated cone. The probe head 420 is used to enter into the internal compartment 211 of one of the aforementioned disposable ear probe covers 200, and the probe head 420 substantially fully fills the internal compartment 211 thereof, however, the disclosure is not limited thereto. The detaching module 430 includes a base 431, a slide assembly 440 and an operation part 500 (FIG. 4). The base 431 is connected to the main body 410 and the probe head 420. The slide assembly 440 is slidably coupled on the base 431. The operation part 500 is connected to the slide assembly 440, and is exposed outwards from the main body 410.

Specifically, the slide assembly 440 includes a first slide part 441, two second slide parts 443, a first spring 446 and two second springs 447. The first slide part 441 is slidably disposed between the probe head 420 and the base 431, and the first slide part 441 is provided with two first inclined surfaces 442 which are opposite to each other. The first slide part 441 is connected to the operation part 500 (e.g., push button or push lever), and is correspondingly moved along with the operation part 500. The first spring 446 is connected to the first slide part 441 and the probe head 420. The second slide parts 443 are respectively disposed on two opposite sides of the first slide part 441, and each of the second slide parts 443 is slidably disposed between the first slide part 441 and the base 431. Furthermore, each of the second slide parts 443 is provided with a second inclined surface 444 and a clasping part 445. Each of the second inclined surfaces 444 confronts one of the first inclined surfaces 442, and complementally contacts with the confronted first inclined surface 442. Each of the clasping part 445 can clasp each side of the flange 220 of the disposable ear probe cover 200. Each of the second springs 447 is connected to one of the second slide parts 443 and the base 431.

Therefore, when the operation part 500 is pushed to correspondingly move the first slide part 441 towards the probe head 420 in a first axial direction (e.g., Z-axis downward), the first slide part 441 and the probe head 420 collectively compress the first spring 446, and by the first inclined surfaces 442 respectively pushing the second inclined surfaces 444, the first slide part 441 is allowed to drive the second slide parts 443 reversely moving away from the first slide part 441 in a second axial direction (e.g., two contrary directions of X-axis) Thus, each of the second slide parts 443 brings its clasping part 445 to correspondingly move away from the probe head 420, and each of the second slide parts 443 and the base 431 collectively compress the corresponding second spring 447.

On the contrary, when not pressing the operation part 500 and the first slide part 441, the first spring 446 restores to push the first slide part 441 away from the probe head 420 in the first axial direction (e.g., Z-axis upward), and each of the second springs 447 restores to push the corresponding second slide part 443 towards the probe head 420 in the second axial direction (e.g., two confronted directions of X-axis) so as to collectively move the clasping parts 445 towards the probe head 420.

In addition, the probe head 420 includes an outer casing 421, an infrared sensor 422, a supportive tube 423 and a support member 424. The outer casing 421 is in a hollow form, and the outer casing 421 is able to be covered by one of the disposable ear probe covers. The supportive tube 423 is in a hollow form, and the supportive tube 423 is received in the outer casing 421 for directly supporting the outer casing 421. The support member 424 is received in the supportive tube 423 for supporting the infrared sensor 422 in the supportive tube 423. One end surface of the outer casing 421 is provided with a first opening 421H. One end surface of the supportive tube 423 is provided with a second opening 423H which is in communication with the first opening 421H. The first opening 421H, the second opening 423H, the supportive tube 423, the support member 424 are coaxial together. The infrared sensor 422 is disposed inside the supportive tube 423, and is sandwiched between the supportive tube 423 and the support member 424. The infrared sensor 422 is aligned with the first opening 421H and the second opening 423H for emitting lights outwardly via the first opening 421H and the second opening 423H.

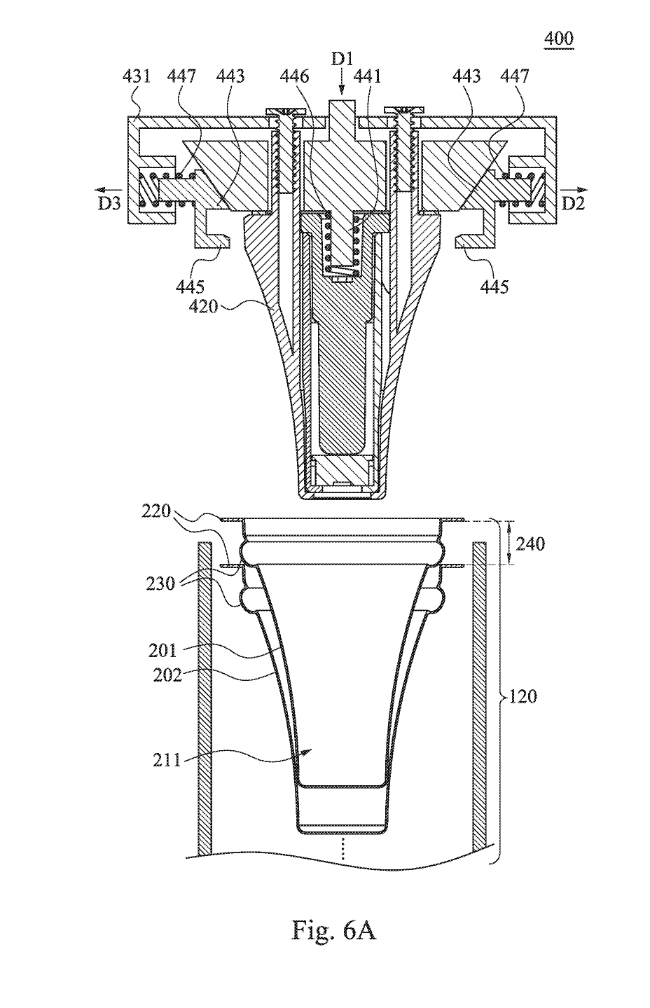

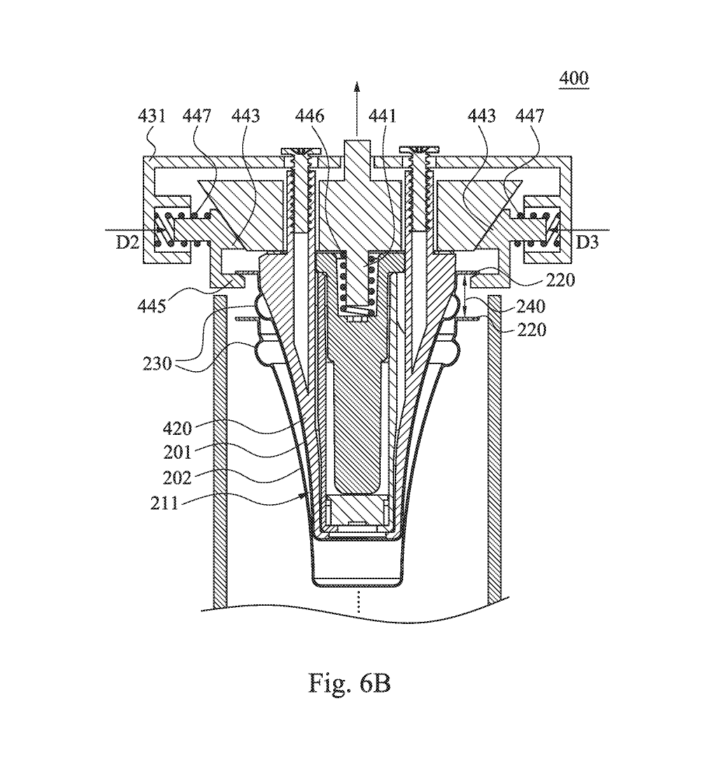

Reference is now made to FIG. 6A and FIG. 6B, in which FIG. 6A is an operational schematic view of the infrared ear thermometer 400 of FIG. 4 pushing the clasping parts 445 away, and FIG. 6B is an operational schematic view of the infrared ear thermometer 400 of FIG. 4 for holding one of the disposable ear probe covers 201. When a user is desired to use the infrared ear thermometer 400 to wear a clean disposable ear probe cover 201 from the superimposed-cover structure 120, as shown in FIG. 6A, the user first moves the operation part 500 (FIG. 4) to correspondingly move the first slide part 441 in a first direction D1 so as to push the clasping parts 445 of the second slide parts 443 away from the probe head 420 along two opposite directions D2, D3, respectively. The user next inserts the probe head 420 into an internal compartment 211 of the last one of the disposable ear probe covers 201 in the first direction D1 while the clasping parts 445 are kept away from the probe head 420. At this moment, the last one of the disposable ear probe covers 201 is arranged between the clasping parts 445. Next, as shown in FIG. 6B, the user releases the operation part 500 (FIG. 4) so that the first spring 446 and the second springs 447 indirectly push the clasping parts 445 to confrontedly approach the last one of the disposable ear probe covers 201 along two opposite directions D3, D2, respectively, and to extend into a void gap 240 defined between two flanges 220 of the last two neighboring disposable ear probe covers 201 so as to grasp the flange 220 of the last one disposable ear probe cover 201 for holding the last one disposable ear probe cover 201.

Therefore, since the flanges of the conventional disposable probe covers are directly stacked with each other, there is no gap existing between any two of the flanges so that a user has to directly touch the disposable probe cover with the hands of the user for removing the clean disposable probe cover away from the remaining disposable probe covers. Thereby, increasing the possibility of the clean disposable probe cover getting contaminated. In the aforementioned embodiments, with the spacer member 230 of the light-transmissive cover body 210, a void gap 240 can exist between any two sleeved disposable ear probe covers 200 so that the infrared ear thermometer 400 of the disclosure can extend into the void gap 240 of the two sleeved disposable ear probe covers 201, 202, and quickly remove the last one of the disposable ear probe covers 201 away from the remaining disposable probe covers without directly touching the disposable probe cover 201 with the hands of the user so as to achieve single-handed operation to replace the disposable ear probe covers. Thereby, not only the possibilities of the clear disposable ear probe covers getting contaminated can be reduced, but also the replacement procedure for the infrared ear thermometer to change the disposable ear probe cover can be simplified, and the replacement time of the disposable ear probe cover can be decreased.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims and their equivalents.

What is claimed is:

1. An ear probe cover set, comprising:
a container; and
a superimposed-cover structure received in the container, shaped as a tubular body formed by a plurality of disposable ear probe covers which are sleeved one another, and each of the disposable ear probe covers comprising:
a light-transmissive cover body provided with an ear-entering end and an opening end which are opposite to each other;
a flange formed on the light-transmissive cover body and surrounding the opening end of the light-transmissive cover; and
a spacer member formed on the light-transmissive cover body,
wherein, in any two neighboring ones of the disposable ear probe covers of the superimposed-cover structure, the spacer member of one of the two neighboring disposable ear probe covers supports the other of the two neighboring disposable ear probe covers such that a void gap is defined between the flanges of the two neighboring disposable ear probe covers.

2. The ear probe cover set of claim 1, wherein the spacer member of each of the disposable ear probe covers is protrusively formed on an outer surface of the light-transmissive cover body.

3. The ear probe cover set of claim 1, wherein the spacer member of each of the disposable ear probe covers is protrusively formed on an inner surface of the light-transmissive cover body.

4. The ear probe cover set of claim 1, wherein the spacer member of each of the disposable ear probe covers is continuously formed for surrounding the light-transmissive cover body.

5. The ear probe cover set of claim 1, wherein the spacer member of each of the disposable ear probe covers is directly connected to the ear-entering end of the light-transmissive cover body.

6. A disposable ear probe cover, comprising:
a light-transmissive cover body provided with an ear-entering end and an opening end which are opposite to each other;
a flange formed on the light-transmissive cover body and surrounding the opening end of the light-transmissive cover; and
a spacer member formed on the light-transmissive cover body,
wherein, when the disposable ear probe cover is sleeved into another disposable ear probe cover which is identical to the disposable ear probe cover, the spacer member of the disposable ear probe cover supports the other disposable ear probe cover such that a void gap is defined between the flange of the disposable ear probe cover and another flange of the another disposable ear probe cover.

7. The disposable ear probe cover of claim 6, wherein the spacer member is protrusively formed on an outer surface of the light-transmissive cover body.

8. The disposable ear probe cover of claim 6, wherein the spacer member is protrusively formed on an inner surface of the light-transmissive cover body.

9. The disposable ear probe cover of claim 6, wherein the spacer member is continuously formed for surrounding the light-transmissive cover body.

10. The disposable ear probe cover of claim 6, wherein the spacer member comprises a plurality of bumps which are separately arranged on the light-transmissive cover body for surrounding the light-transmissive cover body.

11. The disposable ear probe cover of claim 6, wherein the spacer member is directly connected to the ear-entering end of the light-transmissive cover body.

12. A disposable ear probe cover, comprising:
a light-transmissive cover body;
a first surrounding portion protrusively formed on one end of the light-transmissive cover body, and surrounding the light-transmissive cover body; and
a second surrounding portion protrusively formed the light-transmissive cover body, located between the first surrounding portion and the other end of the light-transmissive cover body, and surrounding the light-transmissive cover body,
wherein, when the disposable ear probe cover is sleeved into another disposable ear probe cover which is identical to the disposable ear probe cover, the second surrounding portion of the disposable ear probe cover separates the first surrounding portion of the disposable ear probe cover and another first surrounding portion of the other disposable ear probe cover.

13. The disposable ear probe cover of claim 12, wherein the second surrounding portion is protrusively formed on an outer surface of the light-transmissive cover body.

14. The disposable ear probe cover of claim 12, wherein the second surrounding portion is protrusively formed on an inner surface of the light-transmissive cover body.

15. The disposable ear probe cover of claim 12, wherein the second surrounding portion is continuously formed for surrounding the light-transmissive cover body.

16. The disposable ear probe cover of claim 12, wherein the second surrounding portion is directly connected to the ear-entering end of the light-transmissive cover body.
